(19) United States
(12) Patent Application Publication
(10) Pub. No.: US 2011/0112541 A1
(43) Pub. Date: May 12, 2011

(54) CUTTING GUIDE COMPRISING A MOVEMENT INDICATOR

(75) Inventor: Gordon Dodds, Feldkirchen (DE)

(73) Assignee: DEPUY ORTHOPÄDIE GMBH, Feldkirchen (DE)

(21) Appl. No.: 12/919,834

(22) PCT Filed: Feb. 26, 2009
(86) PCT No.: PCT/EP2009/001586

§ 371 Date: Dec. 17, 2010

(30) Foreign Application Priority Data
Feb. 27, 2008 (GB) 0803510.7

(51) Int. Cl. A61B 17/56

(52) U.S. Cl. 606/87; 606/96

(57) ABSTRACT

An orthopaedic cutting guide assembly and method of cutting a bone are described. A component has a guide for guiding a cutting instrument and at least a first mechanism for securing the component to a bone. A movement indicator has a mechanism for securing the movement indicator to the bone independently of the component. The component includes at least one indicium by which movement of the component relative to the bone can be indicated by relative movement of the movement indicator and the indicium.

The present invention relates to a cutting guide, and in particular to a cutting guide for use in an orthopaedic arthroplasty procedures which can indicate movement of the cutting guide in use.

Cutting guides, including drill guides, are widely used in surgical procedures and are typically mounted to a bone so as to guide the movement of a saw, drill or other cutting instrument, so as to resect the bone or drill a hole at a desired position and/or in a desired direction.

During sawing, in particular, there are a number of problems that arise from the practical problem of the guide holding the saw and the forces required to cut the bone and any clamping action of the bone on the saw. These can result in significant forces being applied to the cutting guide during use, which can cause the cutting guide to move or shift relative to the bone so that the cut is not made at the desired position or in the desired direction.

Computer assisted surgery (CAS) systems provide a way of addressing this problem by navigating the cutting of the bone so that any deviations from the planned position or direction can be identified during the cutting operation. However, not all surgeons are content to use CAS systems and also CAS systems are not widely available. Even if CAS allows a surgeon to realise that they need to modify their cutting action to reduce the forces exerted on a cutting guide, the surgeon must remember their previous motions and carry these into their internal skills-base between operations and make changes the next time they operate. However, this does not allow for tiredness nor special effects such as wedging against anatomy or contact with sclerotic bone during an operation.

The present invention allows a surgeon to determine any movement of a previously aligned guide during and after cutting. Using such cutting guides, the surgeon can more quickly learn to improve their cutting techniques and/or make adjustments to a cut to improve the performance of a surgical procedure.

According to a first aspect of the invention there is provided a cutting guide assembly comprising: a component having a guide for guiding a cutting instrument in use and at least a first mechanism for securing the component to a bone; and a movement indicator having a mechanism for securing the movement indicator to the bone independently of the component, wherein the component includes at least one indicia by which movement of the component relative to the bone can be displayed by relative movement of the movement indicator and the indicia.

Hence, in this way, as the surgeon is using the cutting guide, movement of the guide relative to the bone can be visually determined providing feedback so that the surgeon can change their cutting action and improve the accuracy of the bone cut.

Preferably the cutting guide is an orthopaedic cutting guide. The cutting guide can be for use with a bone of the knee joint, hip joint, shoulder joint or other specific joints of the body. In particular, the cutting guide can be adapted to make a femoral cut or a tibial cut.

The guide can be adapted to receive a number of different types of cutting instruments.

Preferably the guide is a saw guide and the cutting instrument is a saw. The saw can be a powered saw or a manual saw. The saw guide can be in the form of a slot.

The guide can be a drill guide and the cutting instrument can be a drill. The drill guide can be in the form of a circular aperture or hole.

The guide can be adapted for use as a hard tissue cutting guide. The cutting instrument can be a chisel or similar cutting instrument used for shaping hard tissue.

The at least a first mechanism for securing the component to a bone can include a formation or feature for receiving a fastening. For example, the mechanism can include at least two spaced apart apertures for receiving bone pins. A greater number or lesser number of apertures can also be provided. Other fasteners can be used to secure the component to the bone, such as bone screws.

Preferably, the at least one indicium comprises a scale or gradation. The indicium can include an origin or zero movement position.

At least two different indicia can be provided to display movement in the same or different directions. The indicia can be together or separate. The indicia can be at similar positions on the block, e.g. sides, or different positions, e.g. top or bottom and side.

The indicia can be arranged so that they indicate the direction in which any movement of the guide has been in.

The at least one indicium can comprises a further scale. The further scale can be perpendicular to the scale. The scale and further scale can be arranged to display movement in the superior-inferior direction and in the medial-lateral direction. The scale and further scale can be arranged as a cross-hairs.

The cutting guide assembly can further comprise a coupling between the component and movement indicator. The coupling can bear the at least one indicium. The coupling can be a part of the component, e.g. an aperture or formation in the component, or the coupling can be a separate part.

The coupling can be releasably attachable to the component. This helps ease assembly of the cutting guide assembly.

The movement indicator can include a cap. The cap can be releasably attachable to a bone pin.

The component can include an extended aperture within which the movement indicator can be located. The aperture can extend in an inferior-superior direction and/or in a medial-lateral direction.

The cutting guide assembly can further include a marker which is movable relative to the indicium to record the maximal displacement of the component. The marker can be slidable relative to the component. The marker can be slidably received in an aperture. The marker can be dimensioned to loosely receive a part of the movement indicator therein. The marker can have a plurality of parts. Preferably the marker has two parts for each direction of motion that can be detected. The marker can have a friction fit so that its position does not change after maximal displacement.

The cutting guide assembly can further include a motion amplification mechanism. The mechanism can interact with the component and movement indicator to amplify the indicated amount of relative movement of the movement indicator and the indicium. The mechanism can include a lever adapted to amplify the relative motion.

Also provided is a kit of parts comprising the cutting guide assembly aspect of the invention and a positioning guide. The positioning guide can be used to position the movement indicator relative to the component. The positioning guide can comprise a first member configured to attach to the component and/or a second member configured to attach to the movement indicator. The first member and/or second member can be separated by a distance selected to match the size of a coupling for use with the component and movement indicator.

A further aspect of the invention provides a method for cutting a bone, comprising: attaching a component having a cutting guide to the bone, the component having an indicium; attaching a movement indicator to the bone independently of the component and adjacent the indicium; cutting the bone using an instrument guided by the cutting guide; and detecting relative movement between the component and the movement indicator as indicated by relative movement of the movement indicator and the indicia.

An embodiment of the invention will now be described, by way of example only, and with reference to the accompanying drawings, in which:

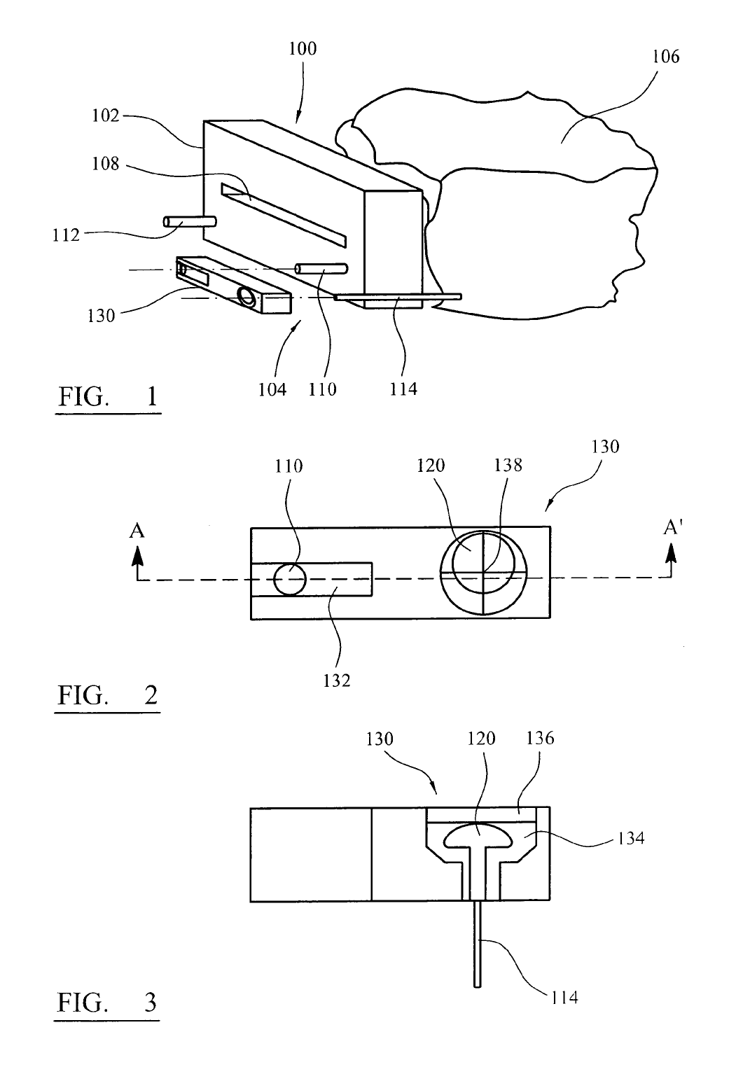

FIG. 1 shows a perspective view of a cutting block assembly according to the invention;

FIG. 2 shows a magnified view of a movement indicator part of the assembly shown in FIG. 1;

FIG. 3 shows a cross section along line AA′ of FIG. 2;

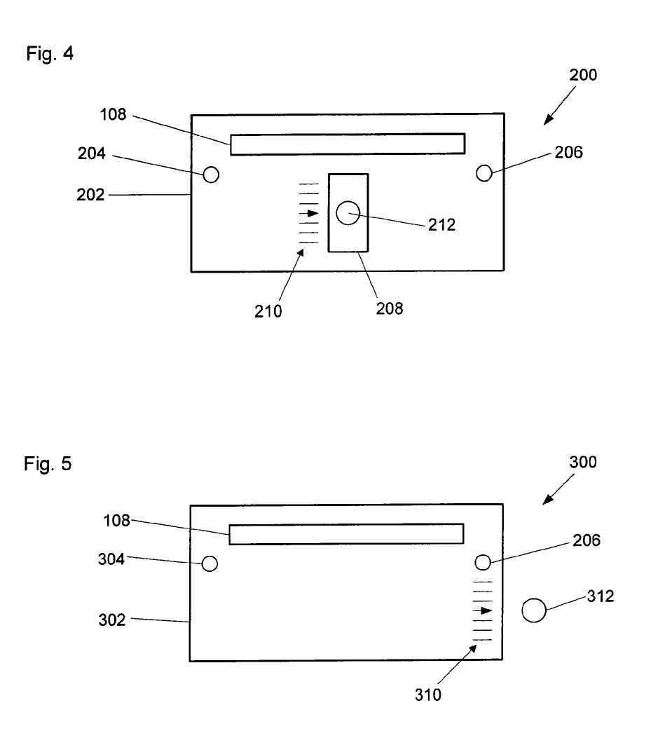

FIG. 4 shows a front view of a second embodiment of the invention;

FIG. 5 shows a front view of a third embodiment of the invention;

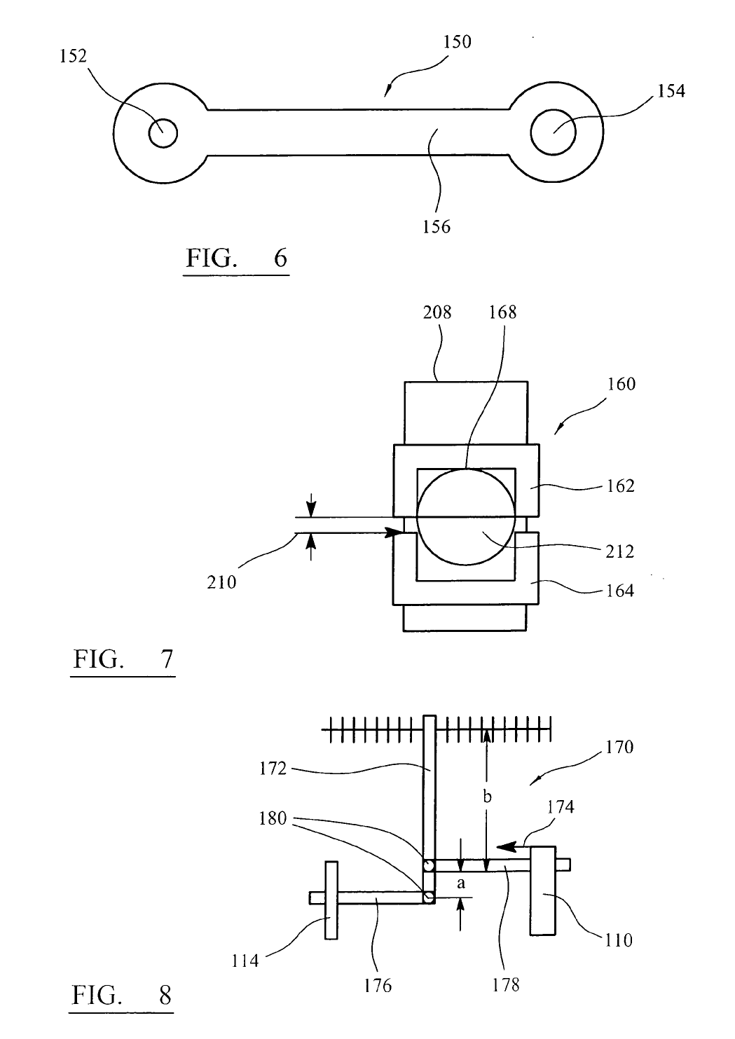

FIG. 6 shows a plan view of a positioning guide component which can be used with the cutting block shown in FIG. 1;

FIG. 7 shows a plan view of a maximum displacement recording component which can be used with the cutting block shown in FIG. 2; and

FIG. 8 shows a plan view of a motion amplification mechanism that can be used with the invention.

Similar items in different Figures share common reference numerals unless indicated otherwise.

With reference to FIG. 1, there is shown a cutting guide assembly 100, including a first component 102 in the form of a cutting block and a movement indicator 104. In the embodiment illustrated in FIG. 1, the cutting block 102 is a tibial cutting block and is shown adjacent the proximal part of a tibia 106. The cutting block 102 includes a cutting guide 108 in the form of a slot for receiving a saw blade in use. Cutting block 102 also includes a pair of spaced apart apertures for receiving bone pins 110, 112 which provide a mechanism for securing the cutting block to the tibia 106.

Assembly 100 also includes a movement indicator 104 which includes a generally mushroom shaped cap 120, a stem of which can be push-fit attached to a free end of a further bone pin 114, that is much thinner than the load bearing pins, so as to secure the movement indicator to the bone 106 independently of the cutting block 102.

Assembly 100 also includes a coupling 130 between the cutting block 102 and the movement indicator 104. An expanded view of coupling 130 is shown in FIG. 2 and a cross section along line AA′ is shown in FIG. 3. Coupling 130 has a generally rectangular form and includes a slot 132 extending along a longitudinal axis. Slot 132 is dimensioned to receive a free end of block pin 110 in a push-fit manner. Coupling 130 also includes a cavity 134 having a generally similar shape to cap 120 and sized to receive cap 120 therein. An upper end of cavity 134 can be closed by a transparent window 136 bearing an indicium in the form of cross hairs 138.

The process of use of the cutting guide assembly 100 will now be described. Initially, the cutting block 102 is mounted on the tibia using conventional bone pins 110, 112 and with the cutting guide 108 at a position on the bone to give a preferred height and direction of tibial cut. Then, a smaller bone pin 114 is inserted in the bone 106 at a position adjacent to the cutting block and so that the cutting block is unlikely to transfer any load thereto during cutting.

FIGS. 6 shows a pin positioning guide component 150 that can be used to help accurately position the bone pin 114 relative to the bone pins 110, 112 of the cutting block. The positioning guide 150 has a first aperture 152 in a first annular end portion with a diameter sized to receive the bone pin 114. The positioning guide 150 has a second aperture 154 in a second annular end portion with a diameter sized to receive bone pins 110 or 112. The two apertures are spaced apart by a body portion 156 of the component so that the apertures are separate by substantially the same distance as the distance between the centre of cavity 134 and the slot 132 of the coupling component.

The positioning guide component is mounted on either of the bone pins 110, 112 using aperture 154 in the second end and then the smaller bone pin 114 is passed through aperture 152 in the first end thereby establishing a preferred distance of separation between bone pin 114 and bone pin 110 or 112 and at which separation then bone pin 114 can be inserted in the bone 106. Hence the positioning guide helps to ensure that the bone pin 114 is positioned at the appropriate position adjacent the cutting guide so that the coupling component 130 will function properly.

The coupling 130 is presented to the non-loaded bone pin 114 and cutting block pin 110, and the cap 120 is push-fit attached to the free end of the non-loaded pin 114 and the cutting block pin 110 is located in adjustment slot 132. The position of the coupling 130 can then be adjusted by sliding the pin 110 in adjustment slot 132 until the centre of the cross hairs 138 is registered with the centre of the cap 120 so as to define the initial zero movement position of the block relative to the movement indicator. The cross hairs are generally aligned with the superior-inferior and medial-lateral directions of the tibia 106.

The surgeon can then insert the saw in cutting guide 108 and begin sawing. During sawing the surgeon can observe the position of the cap 120 through window 136 and any movement of the block away from its initial position is displayed by movement of the cap 120 relative to cross hairs 138. For example, as illustrated in FIG. 2, the cutting block has moved slightly in an inferior direction, possibly owing to excess downward pressure on the block caused by the saw blade. By observing the movement indicator relative to the cross hairs, a surgeon can adjust their sawing action in real time to try and minimise movement of the movement indicator relative to the cross hairs while completing the cut.

The movement indicator, and in particular cap 120, can have a high visibility finish, such as a distinctive colour, to improve its visibility in use.

It will be appreciated that there are a number of variations and modifications which can be made to the cutting guide assembly. For example, the cross hairs 138 can include a scale or gradations.

FIG. 4 shows a second embodiment of a cutting guide assembly 200 according to the invention. The assembly includes a cutting block component 202 including a saw guide 108 and a pair of apertures 204, 206 for accepting bone pins to secure the cutting block to a bone in use. The cutting block component also includes a generally rectangular aperture 208 extending through its body with a scale 210 adjacent thereto. Assembly 200 also includes a movement indicator 212 in the form of a cap similar to cap 120, attached to a free end of a bone pin. Cap 212 and aperture 208 are sized so that cap 212 can freely move within aperture 208 as a result of any relative movement, in a generally inferior-superior direction of the cutting block 202 relative to the bone to which it is attached during use. In an alternate embodiment, aperture 208 has greater width and allows movement of the cap 212 in two dimensions, rather than one, and a cross hairs type scale, similar to that used in the first embodiment, can be used to help indicate relative movement in two dimensions.

Assembly 200 operates similarly to assembly 100 except that it can only display movement of the cutting block in a single direction. Initially cutting block 202 is attached by bone pins to the tibia at a desired position. The movement guide 212 can then be attached to the bone and located in aperture 208 with the movement guide at an initial zero displacement position (indicated by the arrow in FIG. 4) relative to the cutting block. During use, any upward or downward forces exerted on the cutting block by a saw blade may cause cutting block 202 to move relative to the tibia. However, as movement indicator 212 is attached independently of the cutting block to the bone, that movement will be displayed by displacement of the movement indicator 212 relative to the scale 210.

Additionally, the assembly shown in FIG. 4 can include a component for recording or determining the maximum deviation or displacement of the cutting block. FIG. 7 shows a maximum displacement measurement component 160, which can be used with the embodiment shown in FIG. 4. The component 160 comprises two separate, but similarly U-shaped plastic parts 162, 164 forming a slidable marker. An upper part 162 and a lower part 164 each includes a clip type mechanism to the rear by which they are clipped into mating formations adjacent aperture 208, so that each part 162, 164 can move within aperture 208. A collar is use to ensure the marker sits at the correct depth and friction holds the marker in place after it has been displaced by a cutting movement. Hence, when block 202 moves relative to the bone, cap 212 of the pin abuts and engages with, e.g., the upper part 162 at position 164 and can push the upper part 162 to slide along aperture 208 relative to the scale 210. The cap 212 is loosely received within the component 160 and so if the block moves back from its maximal displacement position, the upper component will remain at the position to which it has slid and hence, the maximal displacement, 168 can be read from the position of the upper component 162 relative to the initial zero point of the scale 210.

The embodiment using the maximal displacement indicator is particularly suitable for use in hard-tissue shaping operations that use a chisel or other such tools that are mechanically aligned with a guide. In these situations the maximal displacement indicator 160 is used to show the maximum momentary deviation of the guide from its unstressed position.

FIG. 5 shows a front view of a third embodiment of the cutting guide assembly 300. Again, the cutting guide assembly includes a cutting guide component 302 with a cutting guide 108 for accepting a saw blade in use. First and second apertures 304, 306 are provided in the cutting block for receiving bone pins to attach the cutting block to the tibia in use. The cutting guide component 302 also includes a scale 310 toward a right hand edge. A movement indicator 312 is also provided, comprising a cap similar to cap 120 mounted on a bone pin.

Operation of cutting guide assembly 300 is similar to that described above. Initially, the cutting guide component 302 is attached to the tibia using bone pins. Then the movement indicator is attached adjacent scale 310 via a further bone pin and at a position corresponding to the zero displacement point of the scale 310. Any movement of the cutting block during sawing will be displayed as relative movement between the cutting block and movement indicator 312 as shown by displacement of the movement indicator relative to the zero point on the scale.

In all cases, the zero position for the scale can be adjusted prior to starting cutting, to allow for the particular observation angle that the surgeon will have during cutting. This involves a mix of rotating and translating the coupling in FIG. 1 and in the other cases the scale can be mounted on the block using a slide mechanism to allow the scale position to be adjusted relative to the aperture.

Generally, the invention allows measurement of displacement of the cutting guide with respect to an unstressed alignment feature, the movement indicator, that has previously been set up by the surgeon. The measurement of force must be tolerant of the high speed movement of a saw. By improving the performance of the surgeon during cutting, ultimately the strength of the saw guide retaining devices can be reduced which may result in reduced collateral damage in some patients. Free hand saw guides can be used since the reference that shows deviation from the initial alignment is at the place of cutting. This feed-back is preferable to that given on screen, using a CAS system, since the observed deviations are real time with no processing delays.

If being used with a CAS system to navigate the cutting block attachment position, then the independent pin is placed during an initial approximate alignment, and is attached to the coupling and zeroed when the accurate positioning of the guide on the bone is confirmed by the CAS system.

Free hand sawing can include the use of pin-free systems that are mounted onto the face of the bone with adjustable pins that only pierce the periostrium and a small cone of bone at the tip of the pin. These systems are stable when force is applied centrally and perpendicularly to the circumference defined by the pins and the small, non-load bearing pin will not cause significant tissue damage but enables the instantaneous feed-back of positioning changes without the need for a tracking array to be continually mounted, thereby avoiding the significant resultant obstruction.

Although the invention has been described above with relation to saw guides, invention is applicable to drill guides as well.

Also, although the assembly has been described above in connection with a tibial cutting block, it will be appreciated that the invention can be used for force and motion control for guides on other joints of the body and not exclusively the knee joint.

The movement detection can also use motion amplification mechanics so that small degrees of motion can be converted to a visually discernable motion. FIG. 8 shows an example amplification mechanism 170, whereby the use of a lever 172 amplifies the one degree of freedom motion 174 of the saw-guide pin 110 by a factor of b/a. The lever 172 is attached to linkages 176, 178 which attach to the pins 114, 110, by pivots 180. The pivots 180 can be one degree of freedom, or greater, and several stages of mechanical amplification can be made. The amplification mechanism 170 can be made of plastic or other such material and the pivots 180 can be made by thinning the plastic at the these regions to allow bending or hinge or ball-joints can be used instead.

The direction of indication can be located toward a top or other side of the cutting component, or indeed to any other convenient location.

The two dimensional motion detection can also be expanded to prevent on-pin sliding. Clamping the measurement device onto the load bearing pin prevents the cutting block from sliding off along the pin.

The embodiment illustrated in FIG. 4 is particularly suitable for use in custom patient instrumentation in which the instrumentation is specially designed to fit the patient's anatomy. The movement detection mechanism can be embedded in the custom instrument during manufacturing, for example, by moulding aperture 208 into the body of the customised cutting guide.

Various modifications and changes to the above described embodiments will be apparent from the foregoing description. In particular features of the invention described in connection with a one of the embodiments can be used in connection with other of the embodiments.

1. An orthopaedic cutting guide assembly comprising:
a component having a guide for guiding a cutting instrument in use and at least a first mechanism for securing the component to a bone; and
a movement indicator having a mechanism for securing the movement indicator to the bone independently of the component, wherein the component includes at least one indicium by which movement of the component relative to the bone can be indicated by relative movement of the movement indicator and the indicium.

2. The cutting guide assembly of claim 1, wherein the guide is a saw guide and the cutting instrument is a saw.

3. The cutting guide assembly of claim 1, wherein the guide is a drill guide and the cutting instrument is a drill.

4. The cutting guide assembly of claim 1, wherein the at least a first mechanism for securing the component to a bone includes two spaced apart apertures for receiving bone pins.

5. The cutting guide assembly of claim 1, wherein the at least one indicium comprises a scale.

6. The cutting guide assembly of claim 5, wherein the at least one indicium comprises a further scale and wherein the further scale is perpendicular to the scale and the scale and further scale are arrange to display movement in the superior-inferior direction and in the medial-lateral direction.

7. The cutting guide assembly of claim 1, further comprising a coupling between the component and movement indicator and wherein the coupling bears the at least one indicium.

8. The cutting guide assembly of claim 7, wherein the coupling is releasably attachable to the component.

9. The cutting guide assembly of claim 1, wherein the movement indicator comprises a cap which is releasably attachable to a bone pin.

10. The cutting guide assembly of claim 1, wherein the component includes an extended aperture within which the movement indicator can be located.

11. The cutting guide assembly of claim 1, further including a marker which is movable relative to the indicium to record the maximal displacement of the component.

12. The cutting guide assembly of claim 1, further including a motion amplification mechanism which interacts with the component and movement indicator to amplify the indicated amount of relative movement of the movement indicator and the indicium.

14. A kit of parts comprising:
the cutting guide assembly of claim 1; and
a positioning guide comprising a first member configured to attach to the component and a second member configured to attach to the movement indicator, wherein the first member and second member are separated by a distance selected to match the size of a coupling for use with the component and movement indicator.

15. A method for cutting a bone, comprising:
attaching a component having a cutting guide to the bone, the component having an indicium;
attaching a movement indicator to the bone independently of the component and adjacent the indicium;
cutting the bone using an instrument guided by the cutting guide; and
detecting relative movement between the component and the movement indicator as indicated by relative movement of the movement indicator and the indicia.
